(19) United States
(12) Patent Application Publication
(10) Pub. No.: US 2022/0143100 A1
(43) Pub. Date: May 12, 2022

(54) Adult Liver Progenitor Cells for Treating Acute-On-Chronic Liver Failure

(71) Applicant: Promethera Therapeutics S.A., Mont-Saint-Guibert (BE)

(72) Inventors: Nancy Veulemans, Willebroek (BE); Virginie Barthel, Court-Saint-Etienne (BE); Etienne Sokal, Hoeilaart (BE); Joëlle Thonnard, Gembloux (BE); Nathalie Belmonte, Nice (FR); Yelena Vainilovich, Tervuren (BE)

(21) Appl. No.: 17/441,693

(22) PCT Filed: Mar. 26, 2020
(86) PCT No.: PCT/EP2020/058586

§ 371 Date: Sep. 21, 2021

(30) Foreign Application Priority Data
Mar. 26, 2019 (EP) 19165124.9
Oct. 3, 2019 (EP) 19201342.3

(51) Int. Cl. A61K 35/407; A61K 47/02; A61K 47/42; A61P 1/16

(52) U.S. Cl. CPC A61K 35/407; A61P 1/16; A61K 47/42; A61K 47/02

(57) ABSTRACT

The invention relates to the use of a composition comprising human adult liver-derived progenitor cells, such as heterologous human adult liver-derived progenitor cells (HALPC), for the treatment of a patient who has developed acute-on-chronic liver failure (ACLF) or is at risk of developing ACLF, wherein the treatment comprises a step of administering to said patient an amount of said composition which comprises a dose of 0.25 to 2.5 million said progenitor cells per kg body weight; wherein the composition is substantially free of an effective amount of an anticoagulant, and wherein the patient does not receive any co-treatment with an anticoagulant.

The present invention relates to adult liver progenitor cells that are generated using primary liver cells for use in the treatment of Acute-on-Chronic Liver Failure.

### BACKGROUND OF THE INVENTION

The liver is a key organ in the regulation of body homeostasis and is the site of many vital metabolic pathways. Impairment of only one protein within a complex metabolic pathway could be highly deleterious. The large presence of important liver enzymes substantially increases the risk occurrence of diverse liver diseases. Altogether, 200 different inborn errors of liver metabolism exist, affecting 1 child over 2500 live births. Current treatments, and long-term management, are not efficient enough. Orthotopic liver transplantation (OLT) is highly intrusive, irreversible, limited by shortage of donor grafts and demands state-of-art surgery. Liver cell transplantation (LCT) may exert only short-to-medium term efficacy due to the quality of hepatocyte preparations. Further improvements in tolerance towards cryopreservation, permanent engraftment, and high functionality of the infused cells, would be a major breakthrough (Sokal E M, 2011; Russo F P and Parola M, 2012; Allameh A and Kazemnejad S, 2012; Parveen N et al., 2011).

This improvement are brought by the use of stem or progenitor cells, in particular liver progenitor cells that have been identified in the literature using liver tissues from different organisms, as well as in fetal or adult liver tissues (Schmelzer E et al., 2007; Sahin M B et al., 2008; Azuma H et al., 2003; Herrera M B et al., 2006; Najimi M et al., 2007; Darwiche H and Petersen B E, 2010; Shiojiri N and Nitou M, 2012; Tanaka M and Miyajima A, 2012). Such cells can provide, following the exposure to hepatogenic stimuli in vitro and/or after in vivo administration, cells with morphological and functional features typically associated to hepatic differentiation such as phase I/II enzymatic activities.

These liver progenitor cells or hepatocyte-like cells that are generated from them can be used in cellular transplantation as well as for drug testing in the development of new drugs since they represent a surrogate for primary human hepatocytes in drug metabolism and pharmacological or toxicological in vitro screening (Dan Y Y, 2012; Hook L A, 2012).

WO 2016/030525 discloses specific cell culture conditions allowing the obtention of human adult liver-derived progenitor cells (HALPC) with specific expression profile and improved biological features. Such conditions can be used for producing either cell-based pharmaceutical compositions that can be administered for the treatment of liver diseases, or metabolically and hepato-active cells that can be used for characterizing the efficacy, metabolism, and/or toxicity of a compound.

Acute-on-chronic liver failure (ACLF) is a syndrome characterized by acute decompensation (AD) of chronic liver disease associated with organ failures and high short-term mortality. Among patients with AD not presenting with ACLF (ACLF grade 0), subgroups were identified as being at higher risk of progressing to full ACLF, and thus being at higher mortality risk.

The administration of HALPC to patients has been evaluated in several clinical trials and therapy-induced thrombi have been observed in several patients. Most of these cells express a procoagulant activity linked to tissue factor expression which activates the coagulation cascade and lead to consumption of coagulation factors thus causing severe bleeding. Under these circumstances, it was therefore suggested to control the thrombogenic risk by adding anticoagulants such as heparin and/or bivalirudin to the therapy.

### SUMMARY OF THE INVENTION

The invention relates to the use of a composition comprising human adult liver-derived progenitor cells, such as heterologous human adult liver-derived progenitor cells (HHALPC), also referred as Human Allogeneic Liver Progenitor Cells (HALPC), for the treatment of a patient who has developed or is at risk of developing acute-on-chronic liver failure (ACLF), wherein the treatment comprises a step of administering to said patient an amount of said composition which comprises a dose of 0.25 to 2.5 million of said progenitor cells per kg body weight; wherein the composition is substantially free of an effective amount of an anticoagulant, and wherein the patient does not receive any co-treatment with an anticoagulant.

The inventors have found that such low doses of these progenitor cells are remarkably effective in the treatment of ACLF or conditions that give rise to ACLF, such as Acute Decompensation (AD), even if administered to a patient only once or twice, leading to a dramatic decrease of the patients' bilirubin levels and their MELD scores.

It was also entirely unexpected to discover that human adult liver-derived progenitor cells, such as HALPCs can be administered to ACLF or AD patients according to the invention even in the absence of concomitant anticoagulant therapy. This is surprising since the administration of stem cells such as HALPCs, which are known to affect the coagulation cascade, is generally considered to involve a significant risk of thrombogenesis whose control would normally require the co-treatment with anticoagulants in order to prevent thrombosis or bleeding. However, the inventors identified highly effective amounts of HALPCs that can be safely administered to ACLF patients without significant adverse effects, without co-treatment with anticoagulants.

### DESCRIPTION OF THE DRAWINGS

FIG. 1 depicts the development of MELD scores (left) and Child Pugh scores (right) in ACLF and AD patients treated with HALPCs according to the invention from pre-dose baseline (Pre1) until 3 months after receiving the first dose (3). The bars indicate the means and standard deviations (SD).

FIG. 2 depicts the development of bilirubin levels in ACLF and AD patients treated with HALPCs according to the invention from pre-dose baseline (Pre1) until 3 months after receiving the first dose (3). The bars indicate the means and standard deviations (SD).

### DETAILED DESCRIPTION OF THE INVENTION

The present invention relates to a composition comprising adult human liver-derived progenitor cells for use in the treatment of a patient who has developed or is at risk of developing acute-on-chronic liver failure (ACLF), wherein the treatment comprises a step of administering to said patient an amount of said composition which comprises a dose of 0.25 to 2.5 million of such progenitor cells per kg body weight; wherein the composition is substantially free of an effective amount of an anticoagulant, and wherein the patient does not receive any co-treatment with an anticoagulant. In one of the preferred embodiments, the adult human liver-derived progenitor cells are heterologous human adult liver-derived progenitor cells (HALPC).

ACLF is a condition or syndrome characterized by an acute and sudden deterioration of liver function in a patient with a chronic liver disease, and is associated with hepatic and extrahepatic organ failure and also with high short-term risk of mortality, for example within a period of 28 days from onset (see for example, Hernaez, et al. Gut, 2017 (66) 541-553). ACLF may be characterized for example by acute decompensation (AD) including development of jaundice and prolongation of INR (international normalized ratio), and further complications such as ascites and/or hepatic encephalopathy.

The causes or triggers for ACLF in patients having a chronic liver condition or disease may not always be fully ascertained however, ACLF may be attributed to factors such as and not limited to, any one or combination of, for example, bacterial infection (e.g. sepsis-induced ACLF), viral infection (such as hepatitis B, or other hepatotrophic viruses such as A or E), acute alcoholic hepatitis, surgery, hepatic injury such as from ischaemia, hepatotoxic drugs, and systemic inflammation. Individuals or patients having chronic liver disease and having ACLF or who are at risk for developing ACLF may also optionally be characterized into the following groups: patients with non-cirrhotic chronic liver disease, patients with compensated cirrhosis and patients with decompensated cirrhosis either previously or with contemporaneous cirrhotic decompensation.

In one embodiment, the composition according to the present invention may be used for the treatment of a patient who has developed, or is at risk of developing ACLF, wherein said patient has been or is diagnosed with a liver condition or disease selected from non-cirrhotic chronic liver disease, cirrhosis, compensated cirrhosis, decompensated cirrhosis (DC) or acute decompensated cirrhosis and acute decompensation (AD).

ACLF has been defined by the CLIF (Chronic Liver Failure) research consortium into 3 grades based on retrospectively fitting data on severity linked to mortality score (Moreau et al. 2013). These grades are “grade 1”, defined as single kidney failure or single non-kidney organ failure with an organ dysfunction; “grade 2”, defined as two failing; and “grade 3”, defined as three or more organ failures (4.4% of patients admitted to hospital with acute decompensation).

In general, patients with AD, which are also classified as pre-ACLF or ACLF grade-0, are individuals typically at risk of developing ACLF. For example, such patients may be experiencing worsening of features and symptoms of the diagnosed chronic liver disease within a short period. Pre-ACLF or ACLF grade-0 patients may not yet have developed organ failure, or have only a single organ failure (e.g. liver failure, coagulation, circulatory or respiratory failure) with creatinine less than 1.5 mg/dL and no hepatic encephalopathy, or cerebral failure with a serum creatinine level of less than 1.5 mg/dL. Patients classified as ACLF-1 will have at least one organ failure, either as a single kidney failure without mild or moderate hepatic encephalopathy, or a single non-kidney failure e.g. liver, coagulation circulatory or lung failure that is associated with serum creatinine ranging from 1.5 to 1.9 mg/dL and/or mild-to-moderate hepatic encephalopathy (e.g. grade 1 or 2), or single brain failure with a serum creatinine level of 1.5-1.9 mg/dL. Patients classified as ACLF-2 have two organ failures, and patients classified as ACLF-3 are experiencing three or more organ failures. Higher grade ACLF typically correlates with increased mortality rates.

In one embodiment of the invention, the composition for use is administered to a patient having an ACLF grade of pre-ACLF or ACLF grade-0. In a further embodiment, the composition is administered to a patient having an ACLF grade level selected from ACLF-1 and ACLF-2.

In yet a further embodiment, the invention relates to the use of a composition comprising HALPC for the treatment of a patient with AD and at risk of developing ACLF and/or a cirrhotic patient with ACLF, wherein the treatment comprises a step of administering to said patient an amount of said composition which comprises a dose of 0.25 to 2.5 million HALPC cells per kg body weight; wherein the composition is substantially free of an effective amount of an anticoagulant, and wherein the patient does not receive any co-treatment with an anticoagulant.

As understood herein, a composition which is ‘substantially free of an effective amount of an anticoagulant’ is defined as a composition which comprises no anticoagulant, or a composition where, if it contains some amount of an anticoagulant, then said amount is pharmacologically ineffective, or whose effect on the coagulant status of a patient is negligible. In other words, if an anticoagulant such as heparin is present in the composition, said anticoagulant would be present only as an excipient and would impart no pharmacological effect on the patient as an anticoagulant. In the case of heparin, for example, an effective anticoagulant therapy for an adult patient requires an initial bolus dose of 5,000 I.U. (international units). Thus, an amount of e.g. 500 I.U. would not be considered an effective amount of an anticoagulant. In one embodiment, the composition according to the invention is substantially free of an effective amount of an anticoagulant.

In some embodiments, therefore, the composition comprises less than 5,000 I.U. of heparin per dose. In this context, per dose means that the composition comprises less 5,000 I.U. of heparin in a volume of the composition that contains a single dose of HALPCs. In other embodiments, the composition comprises not more than about 1,000 I.U. of heparin per HALPC dose, such as from about 0.1 I.U. to about 1,000 I.U. of heparin per dose.

In a further embodiment, the composition contains heparin as an excipient at an amount which is not effective, for example at an amount by which the patient receives no more than 10 I.U./kg body weight of heparin with a single dose of HALPC cells according to the invention. Accordingly, the invention provides a composition comprising adult human liver-derived progenitor cells for use in the treatment of a patient who has developed or is at risk of developing acute-on-chronic liver failure (ACLF), wherein the treatment comprises a step of administering to said patient an amount of said composition which comprises a dose of 0.25 to 2.5 million of said progenitor cells per kg body weight; wherein the composition comprises per single dose not more than about 10 I.U./kg body weight of heparin, and wherein the patient does not receive any co-treatment with an anticoagulant.

In yet a further embodiment, the patient receives no more than 500 I.U. of heparin per single dose of HALPC cells according to the invention. Accordingly, the invention provides a composition comprising adult human liver-derived progenitor cells for use in the treatment of a patient who has developed or is at risk of developing acute-on-chronic liver failure (ACLF), wherein the treatment comprises a step of administering to said patient an amount of said composition which comprises a dose of 0.25 to 2.5 million of said progenitor cells per kg body weight; wherein the composition comprises per single dose not more than about 500 I.U. of heparin, and wherein the patient does not receive any co-treatment with an anticoagulant.

As defined herein, a patient who does not receive any co-treatment with an anticoagulant is to be understood as a patient who is not taking any pharmaceutically effective amount of an anticoagulant at the commencement of the treatment with the HALPC composition, or during the period of treatment in accordance with the invention. Said period may be at least 24, or at least 48 hours after the first administration of the composition. In another embodiment, a patient which does not receive any co-treatment with an anticoagulant does not receive any anticoagulant during period of up to about 14, or about 28 days from the first administration of the composition.

The HALPCs of the compositions according to the present invention may be prepared by means of the following method:

In one embodiment, the method of preparing the HALPCs and compositions thereof comprises the steps of:

(a) Disassociating adult liver or a part thereof to form a population of primary liver cells;

(b) Generating preparations of the primary liver cells of (a);

(c) Culturing the cells comprised in the preparations of (b) onto a support which allows adherence and growth of cells thereto and the emergence of a population of cells;

(d) Passaging the cells of (c) at least once;

(e) Isolating the cell population that is obtained after passaging of (d) that are positive for the markers identified in any one of the embodiments described herein.

Concerning Step (a) of the method, the dissociation step involves obtaining a liver or a part thereof that comprises, together with fully differentiated hepatocytes, an amount of primary cells that can be used for producing liver progenitor or stem cells. The term “liver progenitor cell” refers to an unspecialized and proliferation-competent cell which is produced by culturing cells that are isolated from liver and which or the progeny of which can give rise to at least one relatively more specialized cell type. A liver progenitor cell give rise to descendants that can differentiate along one or more lineages to produce increasingly more specialized cells (but preferably hepatocytes or hepato-active cells), wherein such descendants may themselves be progenitor cells, or even to produce terminally differentiated liver cells (e.g. fully specialized cells, in particular cells presenting morphological and functional features similar to those of primary human hepatocytes). The term “stem cell” refers to a progenitor cell capable of self-renewal, i.e., can proliferate without differentiation, whereby the progeny of a stem cell or at least part thereof substantially retains the unspecialized or relatively less specialized phenotype, the differentiation potential, and the proliferation competence of the mother stem cell. The term encompasses stem cells capable of substantially unlimited self-renewal, i.e., wherein the capacity of the progeny or part thereof for further proliferation is not substantially reduced compared to the mother cell, as well as stem cells which display limited self-renewal, i.e., wherein the capacity of the progeny or part thereof for further proliferation is demonstrably reduced compared to the mother cell.

Based on the ability to give rise to diverse cell types, a progenitor or stem cell may be usually described as totipotent, pluripotent, multipotent or unipotent. A single “totipotent” cell is defined as being capable of growing, i.e. developing, into an entire organism. A “pluripotent” cell is not able of growing into an entire organism, but is capable of giving rise to cell types originating from all three germ layers, i.e., mesoderm, endoderm, and ectoderm, and may be capable of giving rise to all cell types of an organism. A “multipotent” cell is capable of giving rise to at least one cell type from each of two or more different organs or tissues of an organism, wherein the said cell types may originate from the same or from different germ layers, but is not capable of giving rise to all cell types of an organism. A “unipotent” cell is capable of differentiating to cells of only one cell lineage.

The liver or part thereof is obtained from a “subject”, “donor subject” or “donor”, interchangeably referring to a vertebrate animal, preferably a mammal, more preferably a human. A part of a liver can be a tissue sample derived from any part of the liver and may comprise different cell types present in the liver. The term “liver” refers to liver organ. The term “part of liver” generally refers to a tissue sample derived from any part of the liver organ, without any limitation as to the quantity of the said part or the region of the liver organ where it originates. Preferably, all cell types present in the liver organ may also be represented in the said part of liver. The quantity of the part of liver may at least in part follow from practical considerations to the need to obtain enough primary liver cells for reasonably practicing the method of the invention. Hence, a part of liver may represent a percentage of the liver organ (e.g. at least 1%, 10%, 20%, 50%, 70%, 90% or more, typically w/w). In other non-limiting examples, a part of liver may be defined by weight (e.g. at least 1 g, 10 g, 100 g, 250 g, 500 g, or more). For example, a part of liver may be a liver lobe, e.g., the right lobe or left lobe, or any segment or tissue sample comprising a large number of cells that is resected during split liver operation or in a liver biopsy.

The cells according to the invention are preferably generated from cells that have been isolated from mammalian liver or part of a liver, where the term “mammalian” refers to any animal classified as a mammal, including, but not limited to, humans, domestic and farm animals, zoo animals, sport animals, pet animals, companion animals and experimental animals, such as, for example, mice, rats, rabbits, dogs, cats, cows, horses, pigs and primates, e.g., monkeys and apes.

More preferably, the liver progenitor cell or stem cell is generated from cells that have been isolated from human liver or a part thereof, preferably human adult liver or a part thereof. The term “adult liver” refers to liver of subjects that are post-natal, i.e. any time after birth, preferably full term, and may be, e.g., at least 1 day, 1 week, 1 month or more than 1 month of age after birth, or at least 1, 5, 10 years or more. Hence, an “adult liver”, or mature liver, may be found in human subjects who would otherwise be described in the conventional terms of “infant”, “child”, “adolescent”, or “adult”. A skilled person will appreciate that the liver may attain substantial developmental maturity in different time postnatal intervals in different animal species, and can properly construe the term “adult liver” with reference to each species.

In an alternative embodiment of the invention, the adult liver or part thereof may be from a non-human animal subject, preferably a non-human mammal subject. Progenitor or stem cells or cell lines, or progeny thereof, derived as described herein from livers of non-human animal or non-human mammal subjects can be advantageously used. By means of example and not limitation, particularly suitable non-human mammal cells for use in human therapy may originate from pigs.

A donor subject may be living or dead, as determined by art-accepted criteria, such as, for example, the “heart-lung” criteria (usually involving an irreversible cessation of circulatory and respiratory functions) or the “brain death” criteria (usually involving an irreversible cessation of all functions of the entire brain, including the brainstem). Harvesting may involve procedures known in the art, such as, for example, biopsy, resection or excision.

A skilled person will appreciate that at least some aspects of harvesting liver or part thereof from donor subjects may be subject to respective legal and ethical norms. By means of example and not limitation, harvesting of liver tissue from a living human donor may need to be compatible with sustenance of further life of the donor.

Accordingly, only a part of liver may typically be removed from a living human donor, e.g., using biopsy or resection, such that an adequate level of physiological liver functions is maintained in the donor. On the other hand, harvesting of liver or part thereof from a non-human animal may, but need not be compatible with further survival of the non-human animal. For example, the non-human animal may be humanely culled after harvesting of the tissue. These and analogous considerations will be apparent to a skilled person and reflect legal and ethical standards.

Liver or part thereof may be obtained from a donor, preferably a human donor, who has sustained circulation, e.g., a beating heart, and sustained respiratory functions, e.g., breathing lungs or artificial ventilation. Subject to ethical and legal norms, the donor may need to be or need not be brain dead (e.g., removal of entire liver or portion thereof, which would not be compatible with further survival of a human donor, may be allowed in brain dead human beings). Harvesting of liver or part thereof from such donors is advantageous, since the tissue does not suffer substantial anoxia (lack of oxygenation), which usually results from ischemia (cessation of circulation).

Alternatively, liver or part thereof may be obtained from a donor, preferably a human donor, who at the time of harvesting the tissue has ceased circulation, e.g., has a non-beating heart, and/or has ceased respiratory functions, e.g., has non-breathing lungs and no artificial ventilation. While liver or part thereof from these donors may have suffered at least some degree of anoxia, viable progenitor or stem cells can also be isolated from such tissues. Liver or part thereof may be harvested within about 24 h after the donor's circulation (e.g., heart-beat) ceased, e.g., within about 20 h, e.g., within about 16 h, more preferably within about 12 h, e.g., within about 8 h, even more preferably within about 6 h, e.g., within about 5 h, within about 4 h or within about 3 h, yet more preferably within about 2 h, and most preferably within about 1 h, such as, within about 45, 30, or 15 minutes after the donor's circulation (e.g., heart-beat) ceased.

The harvested tissues may be cooled to about room temperature, or to a temperature lower than room temperature, but usually freezing of the tissue or parts thereof is avoided, especially where such freezing would result in nucleation or ice crystal growth. For example, the tissue may be kept at any temperature between about 1° C. and room temperature, between about 2° C. and room temperature, between about 3° C. and room temperature or between about 4° C. and room temperature, and may be advantageously be kept at about 4° C. The tissue may also be kept “on ice” as known in the art. The tissue may be cooled for all or part of the ischemic time, i.e., the time after cessation of circulation in the donor. That is, the tissue can be subjected to warm ischemia, cold ischemia, or a combination of warm and cold ischemia. The harvested tissue may be so kept for, e.g., up to 48 h before processing, preferably for less than 24 h, e.g., less than 16 h, more preferably for less than 12 h, e.g., less than 10 h, less than 6 h, less than 3 h, less than 2 h or less than 1 h.

The harvested tissue may advantageously be but need not be kept in, e.g., completely or at least partly submerged in, a suitable medium and/or may be but need not be perfused with the suitable medium, before further processing of the tissue. A skilled person is able to select a suitable medium which can support the survival of the cells of the tissue during the period before processing.

Isolation of progenitor cells or stem cells from a liver or part of a liver is performed according to methods known in the art, for example as described in EP1969118, EP3039123, EP3140393 or WO2017149059 (see Example 1).

A population of liver primary cells is first obtained from disassociating of liver or part thereof, to form a population of primary cells from said liver or part thereof. The term “disassociating” as used herein refers to partly or completely disrupting the cellular organization of a tissue or organ, i.e. partly or completely disrupting the association between cells and cellular components of a tissue or organ to obtain a suspension of cells (a cell population) from the said tissue or organ. The suspension may comprise solitary or single cells, as well as cells physically attached to form clusters or clumps of two or more cells. Disassociating preferably does not cause or causes as small as possible reduction in cell viability. A suitable method for disassociating liver or part thereof to obtain a population (suspension) of primary cells therefrom may be any method well known in the art, including but not limited to, enzymatic digestion, mechanical separation, filtration, centrifugation and combinations thereof. In particular, the method for disassociating liver or part thereof may comprise enzymatic digestion of the liver tissue to release liver cells and/or mechanical disruption or separation of the liver tissue to release liver cells. Small, thin fragments of liver tissues that are obtained by a liver biopsy may be used directly for pursuing cell culture according to the following Step (c) without enzymatic or mechanical disruption.

Methods for disassociating liver or part thereof as above are documented in the literature as the widely used collagenase perfusion technique in two or more steps, which has been variously adapted and modified for performing it with whole livers or segments of liver. The liver tissue is perfused with a divalent cation-free buffer solution, preheated at 37° C., containing a cation-chelating agent (e.g. EDTA or EGTA). Buffer solutions can comprise salt solutions (e.g. HEPES, Williams E medium) or any other balanced salt solution that can also include salts such as NaCl and KCl, among others. This leads to disruption of the desmosomal structures that hold cells together.

The tissue is then perfused with the buffer solution containing divalent cation(s), such as Ca2+ and Mg2+, and matrix-degrading enzymes that act to digest the tissue. The primary liver cells are usually released by gentle mechanical disruption and/or pressing through filters, to mechanically complete the cell dissociation process. Such filters may have sieve sizes that allow passage of cells through about 0.1 mm, 0.25 mm, 0.50 mm, 1 mm or more. A succession of filters with progressively smaller sieve sizes may be used to gradually disassociate the tissue and release cells. The dissociated cells are rinsed with a buffer containing protease inhibitor, serum and/or plasma to inactivate collagenase and other enzymes used in the perfusion process, and then separated from the mixture by pelleting them with low speed centrifugation (e.g. at between 10×g and 500×g). Most of, if not all, viable cells can be pelleted, while dead cells and cell debris are substantially eliminated and subsequently are washed with ice-cold buffer solution to purify the cell suspension. The number and quality of the primary liver cells can vary depending on the quality of the tissue, the compositions of different solutions that are used, and the type and concentration of enzyme. The enzyme is frequently collagenase but also pronase, trypsin, hyaluronidase, thermolysin, and combinations thereof can be used. Collagenase may consist of a poorly purified blend of enzymes and/or exhibit protease activity, which may cause unwanted reactions affecting the quality and quantity of viable cells that can in turn be avoided by selecting enzyme preparations of sufficient purity and quality. Other methods of harvesting primary liver cells may exclude enzymatic digestion techniques and may involve perfusing liver with solutions containing sucrose followed by mechanical disruption.

Concerning step (b) of the method, the population of primary cells as defined and obtained herein by disassociating liver or part thereof may typically be heterogeneous, i.e., it may comprise cells belonging to one or more cell types belonging to any liver-constituting cell type, including progenitor or stem cells, that may have been present in liver parenchyma and/or in the liver non-parenchymal fraction. As used herein, the term “primary cell” includes cells present in a suspension of cells obtained from a tissue or organ of a subject, e.g. liver, by disassociating cells present in such explanted tissue or organ with appropriate techniques.

Exemplary liver-constituting cell types include but are not limited to hepatocytes, cholangiocytes (bile duct cells), Kupffer cells, hepatic stellate cells (Ito cells), oval cells and liver endothelial cells. The above terms have art-established meanings and are construed broadly herein as encompassing any cell type classified as such.

The term “hepatocyte” encompasses epithelial and parenchymal liver cells, including but not limited to hepatocytes of different sizes or ploidy (e.g., diploid, tetraploid, octaploid). A primary cells population may comprise hepatocytes in different proportions (0.1%, 1%, 10%, or more of total cells), according to the method of disassociating liver and/or any methods for fractioning or enriching the initial preparation for hepatocytes and/or other cell types on the basis of physical properties (dimension, morphology), viability, cell culture conditions, or cell surface marker expression by applying any suitable techniques.

The population of primary cells as defined and obtained herein by disassociating liver (or part of it) can be used immediately for establishing cell cultures as fresh primary liver cells or, preferably, stored as cryopreserved preparations of primary liver cells using common technologies for their long-term preservation.

Concerning step (c), the preparation of liver primary cells obtained in step (b) is then cultured directly onto a fully synthetic support (e.g. plastic or any polymeric substance) or a synthetic support pre-coated with feeder cells, protein extracts, or any other material of biological origin that allow the adherence and the proliferation of similar primary cells and the emergence of a population of adult liver progenitor cells having the desired markers, such markers being identified preferably at the level of protein, by means of immunohistochemistry, flow cytometry, or other anti-body based technique. Primary cells are cultured in a cell culture medium sustaining their adherence and the proliferation of and the emergence of a homogenous cell population. This step of culturing of primary liver cells as defined above leads to emergence and proliferation of liver progenitor cells in the culture and can be continued until liver progenitor or stem cells have proliferated sufficiently. For example, culturing can be continued until the cell population has achieved a certain degree of confluence (e.g., at least 50%, 70%, or at least 90% or more confluent). As an example, the cells are cultured for at least 7 days, at least 10, or at least 12 days. In an embodiment, the cells from the primary cell population are cultured within 7 and 12 days. The term “confluence” as used herein refers to a density of cultured cells in which the cells contact one another, covering substantially all of the surfaces available for growth (i.e., fully confluent).

Liver progenitor or stem cells obtained at step (c) can be further characterized by technologies that allow detecting relevant markers already at this stage (that is, before passaging cells as indicated in step (d)), as described in EP3140393 or WO2017149059. Among the technologies used for identifying such markers and measuring them as being positive or negative, Western Blot, Flow Cytometry immunocytochemistry, and ELISA are preferred since these allow marker detection at the protein level even with the low amount of liver progenitor cells that are available at this step.

The isolation or harvesting of liver progenitor cells can then be made based on the confirmation of the cells' identity, i.e. the marker profile, morphology and/or activity. For example, the liver progenitor cells are positive for at least one mesenchymal marker. Mesenchymal markers include but are not limited to Vimentin, CD13, CD90, CD73, CD44, CD29, α-smooth muscle actin (ASMA) and CD140-b. In addition, the liver progenitor cells may secrete HGF and/or PGE2. Moreover, they can optionally be positive for at least one hepatic marker and/or exhibit at least one liver-specific activity. In one embodiment, the cells are positive for at least one hepatic marker and/or exhibit at least one liver-specific activity. For example, hepatic markers include but are not limited to HNF-3B, HNF-4, CYP1A2, CYP2C9, CYP2E1, CYP3A4 and alpha-1 antitrypsin and may also include albumin (ALB). Liver-specific activities may include but are not limited to urea secretion, bilirubin conjugation, alpha-1-antitrypsin secretion and CYP3A4 activity.

In one embodiment, the liver progenitor cells are heterologous human adult liver-derived progenitor cells (HALPC) that express at least one mesenchymal marker selected from CD90, CD44, CD73, CD13, CD140b, CD29, vimentin and α-smooth muscle actin (ASMA), and which also secrete HGF. In a further embodiment, the liver progenitor cells are heterologous human adult liver-derived progenitor cells (HALPC) that express at least one mesenchymal marker selected from CD90, CD44, CD73, CD13, CD140b, CD29, vimentin and α-smooth muscle actin (ASMA), and which also secrete HGF and PGE2.

Concerning Step (d) of the method, primary cells are cultured in a cell culture medium sustaining their adherence and the proliferation of and the emergence of a homogenous cell population that, following at least one passage, is progressively enriched for liver progenitor cells or stem cells. These liver progenitor cells can be rapidly expanded for generating sufficient cells for obtaining progeny having the desired properties, as described for example in EP3140393 or WO2017149059, with cell doubling that can be obtained within 48-72 hours and maintenance of liver progenitor cells having the desired properties for at least 2, 3, 4, 5 or more passages.

The term “passage” or “passaging” is common in the art and refers to detaching and dissociating the cultured cells from the culture substrate and from each other. For sake of simplicity, the passage performed after the first time of growing the cells under adherent culture conditions is herein referred to as “first passage” (or passage 1, P1) within the method of the invention. The cells may be passaged at least one time and preferably two or more times. Each passage subsequent to passage 1 is referred to herein with a number increasing by 1, e.g., passage 2, 3, 4, 5, or P1, P2, P3, P4, P5, etc.

The isolated liver progenitor cells may be plated onto a substrate which allows adherence of cells thereto, and cultured in a medium sustaining their further proliferation, generally a liquid culture medium, which may contain serum or may be serum-free. In general, a substrate which allows adherence of cells thereto may be any substantially hydrophilic substrate. Current standard practice for growing adherent cells may involve the use of defined chemical media with or without addition of bovine, human or other animal serum. These media, that can be supplemented with appropriate mixture of organic or inorganic compounds may, besides providing nutrients and/or growth promoters, also promote the growth/adherence or the elimination/detachment of specific cell types. The added serum, besides providing nutrients and/or growth promoters, may also promote cell adhesion by coating the treated plastic surfaces with a layer of matrix to which cells can better adhere. As appreciated by those skilled in the art, the cells may be counted in order to facilitate subsequent plating of the cells at a desired density.

The term “serum”, as conventionally defined, is obtained from a sample of whole blood by first allowing clotting to take place in the sample and subsequently separating the so formed clot and cellular components of the blood sample from the liquid component (serum) by an appropriate technique, typically by centrifugation. An inert catalyst, e.g., glass beads or powder, can facilitate clotting. Advantageously, serum can be prepared using serum-separating vessels (SST), which contain the inert catalyst to mammals.

The environment in which the cells can be plated may comprise at least a cell medium, in the methods of the invention typically a liquid medium, which supports the survival and/or growth of the isolated liver progenitor cells. The liquid culture medium may be added to the system before, together with or after the introduction of the cells thereto.

The term “cell medium” or “cell culture medium” or “medium” refers to an aqueous liquid or gelatinous substance comprising nutrients which can be used for maintenance or growth of cells. Cell culture media can contain serum or be serum-free.

Typically, the medium will comprise a basal medium formulation as known in the art. Many basal media formulations can be used to culture the primary cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, Medium 199, Waymouth's MB 752/1 or Williams Medium E, and modifications and/or combinations thereof. Compositions of the above basal media are generally known in the art and it is within the skill of one in the art to modify or modulate concentrations of media and/or media supplements as necessary for the cells cultured. A preferred basal medium formulation may be one of those available commercially such as Williams Medium E, IMDM or DMEM, which are reported to sustain in vitro culture of adult liver cells, and including a mixture of growth factors for their appropriate growth, proliferation, maintenance of desired markers and/or biological activity, or long-term storage. Another preferred medium is commercially available serum-free medium that supports the growth of liver progenitor cells, such as e.g. StemMacs™ from Miltenyi.

The term “growth factor” as used herein refers to a biologically active substance which influences proliferation, growth, differentiation, survival and/or migration of various cell types, and may effect developmental, morphological and functional changes in an organism, either alone or when modulated by other substances. A growth factor may typically act by binding, as a ligand, to a receptor (e.g., surface or intracellular receptor) present in cells. A growth factor herein may be particularly a proteinaceous entity comprising one or more polypeptide chains. The term “growth factor” encompasses the members of the fibroblast growth factor (FGF) family, bone morphogenic protein (BMP) family, platelet derived growth factor (PDGF) family, transforming growth factor beta (TGF-beta) family, nerve growth factor (NGF) family, the epidermal growth factor (EGF) family, the insulin related growth factor (IGF) family, the hepatocyte growth factor (HGF) family, the interleukin-6 (IL-6) family (e.g. oncostatin M), hematopoietic growth factors (HeGFs), the platelet-derived endothelial cell growth factor (PD-ECGF), angiopoietin, vascular endothelial growth factor (VEGF) family, or glucocorticoids. Where the method is used for human liver cells, the growth factor used in the present method may be a human or recombinant growth factor. The use of human and recombinant growth factors in the present method is preferred since such growth factors are expected to elicit a desirable effect on cellular function.

Such basal media formulations contain ingredients necessary for mammal cell development, which are known per se. By means of illustration and not limitation, these ingredients may include inorganic salts (in particular salts containing Na, K, Mg, Ca, Cl, P and possibly Cu, Fe, Se and Zn), physiological buffers (e.g., HEPES, bicarbonate), nucleotides, nucleosides and/or nucleic acid bases, ribose, deoxyribose, amino acids, vitamins, antioxidants (e.g., glutathione) and sources of carbon (e.g. glucose, pyruvate, e.g., sodium pyruvate, acetate, e.g., sodium acetate), etc. It will also be apparent that many media are available as low-glucose formulations with or without sodium pyruvate.

For use in culture, basal media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., (3-mercaptoethanol. While many basal media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

Hormones can also be advantageously used in cell culture and include, but are not limited to D-aldosterone, diethylstilbestrol (DES), dexamethasone, estradiol, hydrocortisone, insulin, prolactin, progesterone, somatostatin/human growth hormone (HGH), thyrotropin, thyroxine, L-thyronine, epithelial growth factor (EGF) and hepatocyte growth factor (HGF). Liver cells can also benefit from culturing with triiodithyronine, α-tocopherol acetate, and glucagon.

Lipids and lipid carriers can also be used to supplement cell culture media. Such lipids and carriers can include, but are not limited to cyclodextrin, cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others. Albumin can similarly be used in fatty-acid free formulations.

Also contemplated is supplementation of cell culture medium with mammalian plasma or sera. Plasma or sera often contain cellular factors and components that are necessary for viability and expansion. The use of suitable serum replacements is also contemplated. Suitable sera or plasmas for use in the media as described herein may include human serum or plasma, or serum or plasma from non-human animals, preferably non-human mammals, such as, e.g., non-human primates (e.g., lemurs, monkeys, apes), fetal or adult bovine, horse, porcine, lamb, goat, dog, rabbit, mouse or rat serum or plasma, etc. In another embodiment, any combination of the above plasmas and/or sera may be used in the cell medium.

When passaged, the cultured cells are detached and dissociated from the culture substrate and from each other. Detachment and dissociation of the cells can be carried out as generally known in the art, e.g., by enzymatic treatment with proteolytic enzymes (e.g., chosen from trypsin, collagenase, e.g., type I, II, III or IV, dispase, pronase, papain, etc.), treatment with bivalent ion chelators (e.g., EDTA or EGTA) or mechanical treatment (e.g., repeated pipetting through a small bore pipette or pipette tip), or any combination of these treatments.

A suitable method of cell detachment and dispersion should ensure a desired degree of cell detachment and dispersion, while preserving a majority of cells in the culture. Preferably, the detachment and dissociation of the cultured cells would yield a substantial proportion of cells as single, viable cells (e.g., at least 50%, 70%, 90% of the cells or more). The remaining cells may be present in cell clusters, each containing a relatively small number of cells (e.g., on average, between 1 and 100 cells).

The so detached and dissociated cells (typically as a cell suspension in an isotonic buffer or a medium) may be re-plated onto a substrate which allows the adherence of cells thereto, and are subsequently cultured in a medium as described above sustaining the further proliferation of HALPCs and of HALPC Progeny. These cells may be then cultured by re-plating them at a density of between 10 and 10⁵ cells/cm², and at a splitting ratio between about 1/16 and ½, preferably between about ⅛ and ½, more preferably between about ¼ and ½. The splitting ratio denotes the fraction of the passaged cells that is seeded into an empty (typically a new) culture vessel of the same surface area as the vessel from which the cells were obtained. The type of culture vessel, as well as of surface allowing cell adherence into the culture vessel and the cell culture media, can be the same as initially used and as described above, or may be different. Preferably, cells are maintained onto CellBind® or any other appropriate support that is coated with extracellular matrix proteins (such as collagens, and preferably collagen type I) or synthetic peptides that are acceptable in GMP conditions.

Concerning step (e) above, the isolation of population of HALPCs applies to cells that are positive for the listed markers, further validating the criteria for initially identifying HALPCs at step (c) above but that can be more easily established given the higher amount of cells that are available after passaging.

As used herein, the term “isolated cell” refers generally to a cell that is not associated with one or more cells or one or more cellular components with which the cell is associated in vivo. For example, an isolated cell may have been removed from its native environment, or may result from propagation, e.g., ex vivo propagation, of a cell that has been removed from its native environment.

The terms “cell population” and “population of cells” refer generally to a group of cells. Unless indicated otherwise, the term refers to a cell group consisting essentially of or comprising cells as defined herein. A cell population may consist essentially of cells having a common phenotype or may comprise at least a fraction of cells having a common phenotype. Cells are said to have a common phenotype when they are substantially similar or identical in one or more demonstrable characteristics, including but not limited to morphological appearance, the level of expression of particular cellular components or products (e.g., RNA or proteins), activity of certain biochemical pathways, proliferation capacity and/or kinetics, differentiation potential and/or response to differentiation signals or behavior during in vitro cultivation (e.g., adherence or monolayer growth). Such demonstrable characteristics may therefore define a cell population or a fraction thereof. A cell population may be “substantially homogeneous” if a substantial majority of cells have a common phenotype. A “substantially homogeneous” cell population may comprise at least 60%, e.g., at least 70%, at least 80%, at least 90%, at least 95%, or even at least 99% of cells having a common phenotype, such as the phenotype specifically referred to (e.g., the phenotype of liver progenitor or stem cells of the invention, or to progeny of liver progenitor or stem cells of the invention). Moreover, a cell population may consist essentially of cells having a common phenotype such as the phenotype of liver progenitor or stem cells of the invention (i.e. a progeny of liver progenitor or stem cells of the invention) if any other cells present in the population do not alter or have a material effect on the overall properties of the cell population and therefore it can be defined as a cell line.

Cell count, irrespective of the method which is applied, can be performed on the cell suspension at final harvest, or in the course of preparing the formulation of the pharmaceutical composition to be administered to the patient or during the quality control testing. Any method known in the art can be used, such as the Manual Count Method using Burker Chamber and the Automated Nucleocounter “NC-200”. The aim of these methods is to determine the total amount of cells as well as the amount of the viable ones.

Manual Count Using Burker Chamber

The manual count method using a Burker chamber is based on the Trypan Blue exclusion test.

The cell suspension is diluted with PBS to count between 100 and 200 viable cells per chamber. Trypan blue is added to cell suspension using a ratio 1:1. The cells are counted by microscopy and cell counter. The white cells are viable cells; the blue cells are dead cells. The percentage of viable and dead cells is then calculated. Two cell counts are performed. If the delta Δ between both counts is >15%, a third count is carried out.

Automated Nucleocounter “NC-200”

The Nucleocounter NC-200 1-step provides a high precision automatic cell counter based on fluorescent microscopy and advanced image analysis to perform image cytometric exclusion of dead cells and total cells. The one-step method uses Vial-Cassette™ containing immobilized fluorescent dyes, i.e. Acridine orange and DAPI (4′,6-diamidino-2-phenylindole) that automatically stain the total and dead cell populations respectively.

The cell suspension is diluted to count between 7×10⁵ and 2×10⁶ total cells/ml. The Vial-Cassette™ will pipette a calibrated volume and the NC-200 will automatically determine the total and dead cell concentration and the percentage of viability. All counts are performed in triplicate. The reportable value is the mean out of three valid results of three independently drawn samples. To be valid, total cell count must be between and the coefficient of variation (CV) must not exceed 15.0% for the total cell concentration and for the viability.

In some preferred embodiments, the dosages and dose ranges provided according to the invention are determined according to an automated cell counting method, in particular the automated method described above, using the Nucleocounter NC-200, or an equivalent instrument. For example, a dose of 0.25 to 2.5 million HALPCs per kg body weight should preferably be interpreted as a dosage range wherein the number of cells is determined according to an automated cell counting method, and preferably using an instrument such as the Nucleocounter NC-200 or a technically equivalent alternative thereof. In this context, a technically equivalent alternative means an instrument or cell counting system that leads to substantially equivalent results as the method based on the Nucleocounter NC-200 as described herein, taking into account the variability that is typically observed within and between cell counting methods.

These same preferences apply to other dose ranges, such as a dose of 0.5 to 1.5 million HALPCs per kg body weight, 0.5 to 1.0 million HALPCs per kg body weight, or dosages such as 0.6, 0.8, 1.0, or 1.2 million HALPCs per kg body weight: these are preferably determined by the automated method described above. Moreover, in view of the typical technical variability of the method, a specific number of cells should be understood as that number with a reasonable margin to account for such variability.

The HALPCs, obtained for example as described above, can be used for in vivo administration, for example in the form of a pharmaceutical composition comprising such cells, for treating a patient who has developed or is at risk of developing ACLF. These pharmaceutical compositions can be provided as a HALPC product, optionally combining HALPC with a liquid carrier (e.g. cell culture medium or buffer) that is appropriate for the desired method of treatment, the selected route of administration, and/or storage, as well as in the preferred means for providing such pharmaceutical compositions (e.g. within a kit). Other agents of biological (e.g. antibodies or growth factor) or chemical origin (e.g. drugs, preserving or labeling compounds) that may provide any other useful effect can be also combined in such compositions.

In one embodiment, the HALPC product and/or the pharmaceutical composition comprising the HALPC can be administered systemically, for example by intravenous, intramuscular or intraperitoneal injection, or by intravenous infusion.

Optionally, the administration or the therapeutic use of the HALPC product or composition may comprise the administration or use of another product (which may be, for example a drug, a therapeutic agent, another cell type, or other biological material). An HALPC product may be used in (or for use in) a method of treatment as described herein, wherein the patient is also administered such another product as part of the method. The other product may be administered in combination with the HALPC product, for example as part of the same composition, or separately, simultaneously or sequentially (in any order). The other product may have effects that are compatible, or even synergistic, with the effects (in particular with the therapeutic effects) of an HALPC product.

In one embodiment, the liver progenitor cells comprised in the composition are positive for at least one mesenchymal marker. Mesenchymal markers include but are not limited to Vimentin, CD13, CD90, CD73, CD44, CD29, α-smooth muscle actin (ASMA) and CD140-b. In addition, the liver progenitor cells may secrete HGF and/or PGE2. Moreover, they can optionally be positive for at least one hepatic marker and/or exhibit at least one liver-specific activity. For example, hepatic markers include but are not limited to HNF-3B, HNF-4, CYP1A2, CYP2C9, CYP2E1, CYP3A4 and alpha-1 antitrypsin and may also include albumin (ALB). Liver-specific activities may include but are not limited to urea secretion, bilirubin conjugation, alpha-1-antitrypsin secretion and CYP3A4 activity.

In another embodiment of the present invention, the HALPCs comprised in the composition express at least one mesenchymal marker selected from CD90, CD44, CD73, CD13, CD140b, CD29, vimentin and α-smooth muscle actin (ASMA), and they also secrete HGF.

In another embodiment of the present invention, the HALPCs comprised in the composition express at least one mesenchymal marker selected from CD90, CD44, CD73, CD13, CD140b, CD29, vimentin and α-smooth muscle actin (ASMA), and they also secrete HGF and PGE2.

In another embodiment of the present invention, the HALPCs comprised in the composition express at least one mesenchymal marker selected from CD90, CD44, CD73, CD13, CD140b, CD29, vimentin and α-smooth muscle actin (ASMA), and they optionally also express at least one hepatic marker and/or exhibit a liver-specific activity and/or exhibit a liver-specific activity.

In one embodiment, the cells are positive for at least one hepatic marker and/or exhibit at least one liver-specific activity. For example, hepatic markers include but are not limited to HNF-3B, HNF-4, CYP1A2, CYP2C9, CYP2E1, CYP3A4 and alpha-1 antitrypsin and may also include albumin (ALB). Liver-specific activities may include but are not limited to urea secretion, bilirubin conjugation, alpha-1-antitrypsin secretion and CYP3A4 activity.

In another embodiment of the present invention, the HALPC coexpress at least one mesenchymal marker as described above with respect to step (c); or the marker may be selected from CD90, CD44, CD73, CD13, CD140b, CD29, vimentin and α-smooth muscle actin (ASMA); with one or more hepatic markers selected from alphafetoprotein (AFP), alpha-1 antitrypsin, HNF-4 and MRP2 transporter and optionally the hepatic marker albumin (sometimes also referred to as a hepatocyte marker). They optionally also exhibit a liver-specific activity which may be selected from urea secretion, bilirubin conjugation, alpha-1-antitrypsin secretion and CYP3A4 activity. Moreover, the HALPCs preferably express HGF and PGE-2.

In another or further embodiment of the present invention, the HALPC is measured:
(a) positive for α-smooth muscle actin (ASMA), CD140b and optionally albumin (ALB);
(b) negative for Cytokeratin-19 (CK-19)
(c) and optionally negative for Sushi domain containing protein 2 (SUSD2).

In another embodiment of the present invention, the HALPC cell is measured:
(a) positive for α-smooth muscle actin (ASMA), CD140b and optionally albumin (ALB);
(b) negative for Sushi domain containing protein 2 (SUSD2) and Cytokeratin-19 (CK-19).

In still another or further embodiment of the present invention, the HALPC cell is further measured positive for:
(a) At least one hepatic marker selected from HNF-3B, HNF-4, CYP1A2, CYP2C9, CYP2E1 and CYP3A4 and optionally albumin;
(b) At least one mesenchymal marker selected from Vimentin, CD90, CD73, CD44, and CD29;
(c) At least one liver-specific activity selected from urea secretion, bilirubin conjugation, alpha-1-antitrypsin secretion, and CYP3A4 activity;
(d) At least one marker selected from ATP2B4, ITGA3, TFRC, SLC3A2, CD59, ITGB5, CD151, ICAM1, ANPEP, CD46, and CD81; and
(e) At least one marker selected from MMP1, ITGA11, FMOD, KCND2, CCL11, ASPN, KCNK2, and HMCN1.

In a further embodiment said cells are negative for HLA-DR.

In a further embodiment, the HALPCs are negative for certain markers, such as CD133, CD45, CK19 and/or CD31.

In a further embodiment, the HALPCs may also be measured positive for one or more of the enzymatic activities listed in WO2016/030525, Table 6. In some embodiments, this type of adult liver progenitor cell can be further characterized by a series of negative markers, in particular for one or more of the group consisting of ITGAM, ITGAX, IL1R2, CDH5, and NCAM 1. Additionally, HALPCs may also be measured negative for one or more of the group consisting of HP, CP, RBP4, APOB, LBP, ORM 1, CD24, CPM, and APOC1.

The biological activities, the markers, and the morphological/functional features listed above can be present in HALPCs in different combinations of markers, such as:

(a) positive for α-smooth muscle actin, vimentin, CD90, CD73, CD44, CD29, CD140b, and CYP3A4 activity and optionally albumin; and

(b) negative for Sushi domain containing protein 2, Cytokeratin-19, and CD271.

Further features can be also determined for HALPCs of the above embodiment in any functional and technical combination, for instance by measuring positivity for at least one further marker selected from ATP2B4, ITGA3, TFRC, SLC3A2, CD59, ITGB5, CD151, ICAM1, ANPEP, CD46, and CD81. In some of such embodiments, HALPCs can be measured negative for at least one further marker selected from the group consisting of ITGAM, ITGAX, IL1R2, CDH5, and NCAM1. In some of such embodiments, HHALPCs can be measured negative for at least one of HP, CP, RBP4, APOB, LBP, ORM1, CD24, CPM, and APOC1.

In a further embodiment, the composition according to the present invention is administered to a patient, wherein the patient has, prior to treatment, a MELD score of less than 40, such as in the range from 10 to 40. In a yet further embodiment, the MELD score of the patient is in the range from 13 to 35, and/or has experienced or is experiencing at least one organ failure.

In a further embodiment, the patient's baseline MELD score prior to treatment is between about 20 and 35. In another embodiment, the patient's baseline MELD score prior to treatment is in the range from 17 to 35, or in the range from 17 to 30, respectively. In yet another embodiment, the patient prior to treatment is a candidate for liver transplant, i.e. the patient meets the standard requirements for liver transplantation.

MELD is an acronym for the Model for End-stage Liver Disease scoring system which is used for assessing severity of end-stage liver disease. MELD scores are used in the art to predict mortality, and also to stratify patients (over 12 years old) in respect of the need for a liver transplant. The scoring is based on values of a patient's serum creatinine, bilirubin, INR (international normalized ratio of prothrombin time) and is determined according to the following formula: 9.57×log e (creatinine mg/dL)+3.78×log e (bilirubin mg/dL)+11.2×log e (INR)+6.43. The resulting score is usually rounded to the nearest integer.

In another embodiment, the treatment comprising administration of the composition according to the present invention results in a decrease in MELD score of the patient. For example, the MELD score may be decreased by 10% or more in the course of the treatment. In a further embodiment, the MELD score decreases by at least 20%, preferably within a period of 28 days, or alternatively, preferably within a period of about 1 month, or 3 months after administration of the first dose of the composition. In further embodiments, the MELD score is decreases by at least 25%, or by at least 30%, respectively. The percentage decrease of the MELD score is determined by a comparison of the MELD score of the patient having received the treatment compared to the MELD score obtained from the patient before treatment, i.e. before the first infusion or provision of the composition to the patient.

Moreover, the effect of the treatment may be indicated by an improvement of the Child-Pugh score of the patients. The Child-Pugh score, also referred to as Child-Turcotte-Pugh score or Child Criteria, is used to assess the prognosis of chronic liver disease. In some embodiments, the HALPC treatment according to the present invention is administered to a patient, in particular to a patient suffering from ACLF, who has, prior to treatment, a Child-Pugh score in the range from about 5 to about 15, or from about 6 to about 14, respectively. In some embodiments, the Child-Pugh score of patients treated according to the invention decreases by at least about 10% within one month from the start of the treatment according to the invention, i.e. from the pre-dose baseline value to the Child-Pugh score estimated one month after the dosing (or first dosing, respectively) of the HALPCs. In further embodiments, a decrease of the Child-Pugh score of at least about 10% occurs within two months, or within three months, after the (first) administration of the cells. In yet further embodiments, the Child-Pugh score of patients treated according to the invention decreases by at least about 20% within two months, or within three months, after the (first) administration of the cells.

In a further embodiment, the composition is administered to a patient exhibiting a total bilirubin serum concentration of at least 5 mg/dL prior to the commencement of treatment, i.e. prior to first infusion of the composition. In a further embodiment, the total bilirubin serum concentration prior to the first infusion is at least about 6 mg/dL.

As mentioned, the treatment according to the present invention comprises the administration of a dose of 0.25 to 2.5 million HALPC cells per kg body weight. In one of the preferred embodiments, the dose administered to the patient is in the range from about 0.25 to about 2 million cells per kg. In another preferred embodiment, the dose is in the range from about 0.25 to about 1.5 million cells per kg, or in the range from about 0.25 to about 1.25 million cells per kg, or in the range from about 0.5 to about 1.2 million cells per kg, for example about 0.5, 0.6, 0.64 to 0.8, 1.0, or 1.2 million cells per kg, respectively. It was surprisingly found by the inventors that such relatively low doses of HALPCs, in particular of HALPCs having some or all of the preferred features as described above relating to the markers that they express, are remarkably effective in improving the liver function as well as the overall status of patients, while at the same time avoiding the side effects typically associated with the administration of significant amounts of stem cells. In some embodiments, these ranges are determined using the automated cell counting method as described above.

In a further embodiment, the composition administered to the patient comprises a dose of about 0.25 to about 1.5 million cells per kg, about 0.25 to about 1.25 million cells per kg, about 0.5 to about 1.2 million cells per kg, or about 0.5 to about 1 million HALPC cells per kg body weight.

In a particular embodiment, the invention relates to a composition comprising HALPC for use in the treatment of a patient with AD and at risk of developing ACLF and/or a cirrhotic patient with ACLF, wherein the treatment comprises a step of administering to said patient an amount of said composition which comprises a dose of about 0.25 to about 1.5 million cells per kg, about 0.25 to about 1.25 million cells per kg, about 0.5 to about 1.2 million cells per kg, or about 0.5 to about 1 million HALPC cells per kg body weight; wherein the composition is substantially free of an effective amount of an anticoagulant, and wherein the patient does not receive any co-treatment with an anticoagulant.

In further embodiments, the composition for use according to invention may be administered to a patient who has developed or is at risk of developing ACLF. The composition comprises a dose of about 0.25 million HALPC cells per kg body weight, about 0.5 million HALPC cells per kg body weight, or about 1 million HALPC cells per kg body weight of the patient. In another embodiment, the composition comprises a dose of no more than about 1.5 million cells per kg body weight, or no more than about 2.5 million cells per kg body weight.

According to a further embodiment, the dose administered to the patient is from about 50 to about 200 million HALPCs, or from about 50 to about 150 million HALPCs, such as about 100 million HALPCs.

In any of the preceding embodiments, the composition comprising the HALPCs may be administered to the patient in the form of a sterile liquid.

Said sterile liquid may be prepared from a reconstituted suspension of HALPC cells, prepared for example by the dilution of a thawed concentrated HALPC cell suspension with a sterile diluent, such as a sterile aqueous solution, optionally comprising excipients such as pH-modifiers and/or human serum albumin, which is physiologically compatible with the patient and adapted for intravenous infusion.

In one embodiment, the composition is administered via intravenous infusion, optionally using a peripheral catheter. Alternatively, the composition may be administered to the patient through a central line.

The volume and concentration of the composition in the form of a sterile liquid comprising the HALPC cells is preferably adapted for intravenous infusion. In an embodiment, the composition may be administered to the patient in the form of a sterile liquid comprising, after final adjustment, the HALPC cells at a concentration of up to about 10 million cells, per mL, and particularly from about 0.5 to 5 million cells per mL. In another embodiment, the patient may be administered a sterile liquid composition with concentration of 0.5 to 2 million HALPC cells per mL, such as about 1 million HALPCs per mL. Alternatively, the cell concentration of the composition may be in the range from about 1 to about 5 million cells per mL, or from about 2 to 5 million cells per mL, respectively. These final cell concentrations may be obtained by appropriately diluting a more concentrated HALPC composition.

The volume of the composition which is administered per infusion to a patient is preferably adapted in accordance with the patient's body weight. In one embodiment, the volume of the composition administered per infusion after final adjustment of the cell concentration may be in the range from about 5 to about 500 mL, and preferably from about 10 to about 200 mL, or from about 20 to about 150 mL, respectively.

In yet another embodiment, the composition used for carrying out the invention is a sterile liquid composition which is intravenously infused to the patient at an infusion rate of about 0.1 to about 5 mL per minute, or at a rate of about 0.5 to 2 mL per minute, respectively. It is also preferred that the infusion rate is selected such that an overall infusion time of not more than about 4 hours, or even not more than about one hour, is required for administering a single dose. For example, the composition may be intravenously infused to the patient at an infusion rate of about 1 mL per minute. Further preferred is an infusion rate of about 1 to 2 mL per minute, such as about 1.5 mL per minute. As used herein, the term infusion rate should be understood to include an average infusion rate, such as the total volume of the composition infused per dosing divided by duration of the infusion.

In a further preferred embodiment, the infusion rate is selected in the range of about 0.5 to about 10 million cells per minute, or from about 1 to about 7.5 million cells per minute, respectively.

The composition may be administered to the patient using an infusion bag, such as a 150 mL mixing and infusion bag made of ethylenevinylacetate (EVA), e.g. MIB150 (by Hegewald or Hemedis) which holds the composition having its final concentration. The infusion bag may be connected to a tube, such as a blood administration tubing and filter set (filter pore size about 200 μm) and a flow regulator.

In another preferred embodiment, the composition is administered using a syringe pump. An example of a suitable device is the CA-700 ambulatory syringe pump (Canafusion Technology Inc.). However, any other syringe pump compatible with a syringe having the desired internal volume capacity (e.g. 50 mL) and an adjustable flow rate may also be used. The syringe pump should preferably be mounted such that the syringe has a vertical orientation. The inventors have found that, at the preferred infusion rates, the vertical orientation leads to a more homogeneous delivery of the HALPCs to the patient over the infusion time and makes agitation of the composition during the infusion process unnecessary. Accordingly, in a preferred embodiment, the composition is administered to a patient with a vertically mounted infusion pump at a rate of about 0.1 to about 5 mL per minute, or at a rate of about 0.5 to 2 mL per minute, such as 1.5 mL per minute.

The inventors have further discovered that a particularly effective treatment is achieved by a dosing regimen comprising at least two dosings of the HALPCs. Accordingly, a further embodiment of the invention relates to a composition comprising HALPC for use in the treatment of a patient who has developed or is at risk of developing ACLF, wherein:

a) the treatment comprises a step of administering a first amount of said composition comprising a dose of 0.25 to 2.5 million HALPC cells per kg body weight, wherein said composition is substantially free of an effective amount of an anticoagulant, and wherein the patient does not receive any co-treatment with an anticoagulant; and wherein

b) the treatment further comprises a step of administering to said patient a second amount of said composition, said second amount comprising a second dose of 0.25 to 2.5 million HALPC cells per kg body weight and wherein said composition is substantially free of an effective amount of an anticoagulant, and wherein the patient does not receive any co-treatment with an anticoagulant; and

wherein said second amount is administered 5 to 21 days after the first amount.

The inventors have surprisingly found that the time interval of 5 to 21 days between the first and the second dosing contributes substantially to the tolerability of the treatment, and that shorter intervals should be avoided. In particular, the occurrence and severity of adverse events such as bleeding or thrombosis in the patients can be significantly reduced.

In one embodiment, the time interval between administration of the first and second amount of composition is greater than 4 days, such as from 5 to 21 days. In another embodiment, the second amount of the HALPC composition is administered 6 to 8 days after the first amount. In yet a further embodiment, the second amount of composition is administered 7 days after said first amount.

In a further embodiment, the second amount administered to the patient comprises a dose of 0.5 to 1 million HHLAPC cells per kg body weight. In another embodiment, the second amount of the composition administered to the patient comprises a dose of 1 to 2.5 million HHLAPC cells per kg body weight.

In yet a further embodiment, the first and second amount of the composition administered to the patient may be the same. Alternatively, the first amount of the composition administered to the patient may be independently selected from the second amount of the composition.

The invention may further be described as a method of treating a patient who has developed or is at risk of developing ACLF, wherein the treatment comprises a step of administering to said patient an amount of a composition which comprises a dose of 0.25 to 2.5 million human adult liver-derived progenitor cells, such as HALPC cells, per kg body weight of the patient; wherein the composition is substantially free of an effective amount of an anticoagulant, and wherein the patient does not receive any co-treatment with an anticoagulant.

Moreover, the present invention relates to the use of human adult liver-derived progenitor cells, such as HALPC, for the manufacture of a medicament for the treatment and/or prevention of ACLF, wherein the treatment comprises a step of administering to a patient an amount of said composition which comprises a dose of 0.25 to 2.5 million of such progenitor cells per kg body weight, wherein the composition is substantially free of an effective amount of an anticoagulant, and wherein the patient does not receive any co-treatment with an anticoagulant.

### Further Definitions

The term “adult human liver-derived progenitor cells” is used synonymously with “human adult liver-derived progenitor cells” or “Human allogeneic liver progenitor cells”; “heterologous human adult liver-derived progenitor cells”, abbreviated as “HHALPC” or “HHALPCs” or “HALPC” or “HALPCs” represent a specific type of adult human liver-derived progenitor cells, obtainable as described herein-above. A person skilled in the relevant technical field will understand that these cells have been commonly labelled as “heterologous”, even though derived from human livers. Therefore, these cells could also be labelled as “allogeneic” rather than “heterologous”, to convey the connotation that they are derived from different individuals of the same species as those who will receive the cells for treatment.

The term “in vitro” as used herein denotes outside, or external to, animal or human body. The term “in vitro” as used herein should be understood to include “ex vivo”. The term “ex vivo” typically refers to tissues or cells removed from an animal or human body and maintained or propagated outside the body, e.g., in a culture vessel.

The term “pharmaceutically acceptable carrier” as used herein, refers to a carrier or a diluent that does not cause significant irritation to a subject and does not abrogate the biological activity and properties of the administered composition. Examples, without limitations, of carriers are propylene glycol, saline, emulsions and mixtures of organic solvents with water.

The term “sufficient amount” means an amount sufficient to produce a desired and measurable effect, e.g., an amount sufficient to alter a protein expression profile.

The term “therapeutically effective amount” is an amount that is effective to ameliorate a symptom of a disease. A therapeutically effective amount can be a “prophylactically effective amount” as prophylaxis can be considered therapy.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

The term “allogeneic” as used herein means that the donated material comes from different individual than the recipient. Allogeneic stem cell transplantation refers to a procedure in which a person receives stem cells from a genetically similar, but not identical, donor.

The term “fibrosis” as used herein refers to the formation of excess fibrous connective tissue in an organ or tissue in a reparative or reactive process.

The term “liver fibrosis” refers to the accumulation of interstitial or “scar” extracellular matrix after either acute or chronic liver injury. Cirrhosis, the end-stage of progressive fibrosis, is characterized by septum formation and rings of scar that surround nodules of hepatocytes. Typically, fibrosis requires years or decades to become clinically apparent, but notable exceptions in which cirrhosis develops over months may include pediatric liver disease (e.g. biliary atresia), drug-induced liver disease, and viral hepatitis associated with immunosuppression after liver transplantation.

The following examples serve to illustrate the invention, however should not to be understood as restricting the scope of the invention.

### EXAMPLES

### Example 1: Preparation of HALPCs

HALPCs were prepared as described in EP 3140393 or WO2017/149059 from livers of healthy cadaveric or non-heart beating donors. Briefly, liver cell preparations are re-suspended in Williams' E medium supplemented with 10% FBS, 10 mg/ml INS, 1 mM DEX. The primary cells are cultured on Corning® CellBIND® flasks and cultured at 37° C. in a fully humidified atmosphere containing 5% CO₂. After 24 hours, medium is changed in order to eliminate the non-adherent cells and thereafter renewed twice a week, whereas the culture is microscopically followed every day. Culture medium is switched after 12-16 days to high glucose DMEM supplemented with 9% FBS. A cell type with mesenchymal-like morphology emerges and proliferates. When reaching 70-95% confluence, cells are trypsinized with recombinant trypsin and 1 mM EDTA and re-plated at a density of 1-10×10³ cells/cm². At each passage, cells were trypsinized at 80-90% confluency.

Testing of the cells confirmed that they expressed, inter alia, the following markers: CD90, CD73, Vimentin, and ASMA. The cells were also tested and found negative, or exhibit very low expression, with respect to the following markers: CD133, CD45, CK19 and CD31. The HALPCs were filled into vials in aliquots of 5 mL, each comprising 10 to 50×10⁶ cells/ml, equivalent to 50 to 250×10⁶ cells per vial, and frozen.

### Example 2: Administration of HALPCs to Patients (Interim Results)

Eight patients with confirmed acute-on-chronic liver failure (ACLF) and seven patients with acute decompensation (AD), with risk of developing ACLF, were treated with HALPCs prepared according to Example 1, using the dosing regimen according the present invention. The cells were counted using the manual method described above. The MELD score of the patients prior to treatment ranged from 18 to 35, with an average of about 27. The total bilirubin serum concentration of each patient was higher than 6 mg/dL (≥100 umol/L); between the patients, it ranged from about 7 to about 43 mg/dL with an average of about 22 mg/dL. All patients received standard medical treatment (SMT) as required by their clinical status, but no concomitant anticoagulant therapy.

For each administration of HALPCs, a vial with the cells prepared according to Example 1 was thawed and diluted with 45 mL of a sterile liquid carrier which contained a sodium bicarbonate and human serum albumin along with pharmacologically ineffective trace amounts of heparin (not higher than 500 I.U./patient) as an excipient. A volume of the composition which was calculated to contain the designated dose of cells was administered by intravenous infusion.

The first enrolled patient did not receive the cells because of technical issue. Three of the fifteen patients received HHALPCs at a single dose of 0.25 million cells/kg body weight, and nine patients received a dose of 0.5 million cells/kg. To seven of the patients who had received 0.5 million cells/kg, a second dose of 0.5 million cells was administered seven days after the first administration.

In spite of the low doses of cells that were administered, it was found that these dosing regimens constituted highly effective therapeutic interventions for the patients in terms of improved liver function and systemic inflammation. Bilirubin levels decreased substantially, as well as the MELD scores of the patients. Moreover, the improvement of the patients was sustainable throughout the entire follow-up period: For the patients with transplant-free survival at M3 (month 3 after the commencement of the treatment), bilirubin levels had decreased by approximately 60-80%, and the MELD score dropped by 40-55%.

In addition, two patients (both ACLF) were treated as described above, except that a single dose of 1.0 million cells per kg body weight was administered per dosing. One patient showed a clear improvement of bilirubin and MELD score immediately after treatment; the other patient also showed treatment effects, even though with some delay. All experienced adverse events related to the underlying diseases and comorbidities.

Also remarkable was the fact that the HALPCs were successfully administered to the patients even in the absence of concomitant anticoagulant therapy. This is particularly surprising since the administration of stem cells in general, including HALPCs, which express tissue factor that can activate the coagulation cascade, is typically believed to involve an increased risk of thrombogenesis, for which reason the co-treatment with anticoagulants is normally considered necessary in order to prevent thrombosis or bleeding. However, these results show that highly affective amounts of HALPCs can be safely administered to patients with ACLF or at risk of developing ACLF; even though these patients are substantially compromised, they tolerated the treatment very well, without significant adverse effects that were related to the cell therapy. In particular, no clinically significant drop in platelets, fibrinogen, or coagulation factors were observed. Any reported adverse events (AEs) which were observed for these patients were related to the underlying diseases and comorbidities.

It is noted that this Example 2 represents interim results from a clinical study whose complete results are provided as Example 3 below.

### Example 3: Administration of HALPCs to Patients (Complete Results)

The clinical trial of Example 2 was continued until 22 patients in total were subjected to the treatment according to the invention. The cell counts and dosages of the present Example are provided as determined by the automated method described above, using a Nucleocounter NC-200. In summary, the patients were treated as follows:

One patient received no cells due to a technical issue. Three patients received one infusion of about 0.6 million cells/kg. Three further patients received two infusions of about 0.6 million cells/kg at an interval of about 7 days. Three further patients received one infusion of about 0.8 million cells/kg. Four patients received one infusion with about 1.2 million cells/kg. Eight patients received two infusions of about 1.2 million cells/kg at an interval of about 7 days. In all cases, the infused cells were comprised in compositions that contained only pharmacologically insignificant amounts of heparin, i.e. not more than 10 I.U. per kg body weight. None of the patients received anti-coagulant co-medication.

In result, the HALPC dosages administered to the patients appeared to be well tolerated. The adverse events that were reported for these patients were related to the underlying diseases and comorbidities.

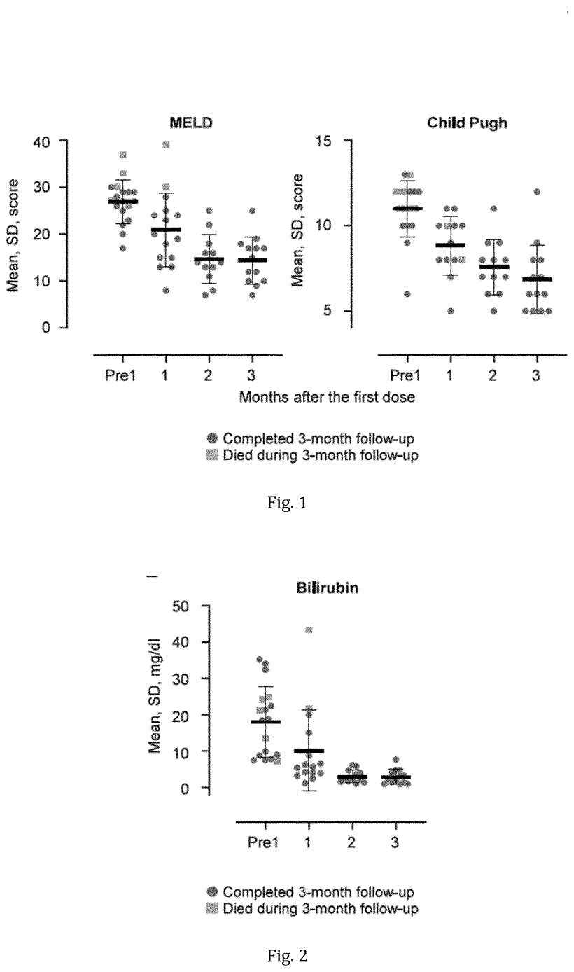

In terms of efficacy, the averages of the prognosis scores for the surviving patients were generally lower at Month 3 than at Day 1, and no patient at Month 3 had ACLF. The improvement in the prognosis scores is illustrated by the development of the MELD score (particularly relevant for transplant prioritization) and the Child-Pugh score (particularly relevant for evaluating the mortality risk) in the patients, as shown in FIG. 1. At Months 1, 2 and 3, the average MELD scores of 21 (standard deviation (SD)=7.9), 15 (SD=5.2) and 14 (SD=5.0), respectively were lower than at baseline (27; SD=4.2). At Month 3, the MELD score was lower than the respective baseline for 12/13 (92%) surviving patients, and the MELD score for 8/13 patients (61%) ≤15. At Months 1, 2 and 3, the average Child-Pugh scores of 8.8 (SD=1.7), 7.6 (SD=1.6) and 6.8 (SD=2.0), respectively were lower than at baseline (11; SD=1.6). At Month 3, the Child-Pugh score was lower than the respective baseline for 12/13 (92%) surviving patients, and the Child-Pugh score for 7/13 (53%) patients ≤6.

Hematology and serum biochemistry values suggested stabilization or improvements to liver function over the 3-month period, including those values that contributed to the MELD and Child-Pugh scores. As shown in FIG. 2, for example, the averages and standard deviations of the bilirubin values of 3.0 mg/dl (SD=1.8) and 2.9 mg/dl (SD=2.0), at Month 2 and Month 3, respectively, were much lower than at baseline (18 mg/dl; SD=9.6), even though the averages remained above the normal reference range (0-1.2 mg/dl). Creatinine and sodium levels appeared to be generally stable over the active study period suggesting that any kidney conditions did not worsen.

In summary, the results obtained in this study showed improvement of liver function and systemic inflammation post infusion. The clinically significant MELD and bilirubin improvement is considered as an encouraging sign of efficacy.

### Example 4: Efficacy and Safety of HALPCs in Patients with ACLF

A randomized, placebo-controlled, double blind, multi-centre Phase IIb study is conducted to evaluate the efficacy and safety of HALPCs in patients with Acute on Chronic Liver Failure (ACLF). Patients recently diagnosed with ACLF grade 1 or 2 will be proposed to undergo the screening procedures to participate in the study. ACLF grading will be based on the CLIF Organ Failure (CLIF-OF) score. Patients will be at least 18 years old and have a bilirubin value of at least 5 mg/dL. Moreover, the patients to be enrolled in the study will have a MELD score of not higher than 35 and free of an underlying cirrhosis due to biliary disease or autoimmune hepatitis.

The patients participating in the study will then be randomised and allocated either to receive the best standard of care treatment plus a placebo, or the best standard of care plus two infusions of HALPCs at an interval of about 1 week, each infusion containing an HALPC dose of about 1 million cells per kg body weight. The HALPC infusions will be administered as compositions that do not comprise any pharmacologically relevant amounts of anticoagulants, in particular not more than 10 I.U./kg heparin per infusion.

An evaluation period of 3 months post-infusion will follow the treatment regimen in which the safety and efficacy data will be obtained from the patients.

In the light of the previously obtained clinical data (see Examples 2 and 3), it is expected that this further study will confirm the efficacy of HALPCs in patients with ACLF using a dosage regimen according to the present invention. More specifically, it will also demonstrate the efficacy of 2 infusions (i.v.) of HALPCs, each containing 1.0 million of cells/kg, administered at a 7-day interval, in terms of a positive effect on the overall survival proportion of the patients 90 days post-first infusion.

### Comparative Example

Two patients suffering from ACLF were treated as described in Example 2, except that a single dose of 250 million HALPCs was administered per dosing, i.e. approx. 4-5.5 million cells per kg body weight. One of the patients received a second dose of 250 million cells four days after the first dose. The patients experienced adverse effects that are related to the treatment: Both patients experienced a strong drop in coagulation factors with severe peripheral bleeding, including severe epistaxis. One patient bled at the insertion side of the transjugular biopsy.

Without wishing to be bound by theory, it is believed that a possible mechanism of action leading to severe bleeding post cell infusion could be related to the activation of the coagulation cascade by the cells, possibly due to an expression of tissue factor by the HALPCs, leading to a consumption of coagulation factors in patients having a limited reserve of these factors due to the liver insufficiency; the decrease in coagulation factors subsequently led to bleeding.

1. A composition comprising adult human liver-derived progenitor cells for use in the treatment of a patient who has developed or is at risk of developing acute-on-chronic liver failure (ACLF), said cells are heterologous human adult liver-derived progenitor cells (HALPC) that express at least one mesenchymal marker selected from CD90, CD44, CD73, CD13, CD140b, CD29, vimentin and α-smooth muscle actin (ASMA), wherein the treatment comprises a step of administering to said patient an amount of said composition which comprises a dose of 0.25 to 2.5 million of said progenitor cells per kg body weight; wherein the composition is substantially free of an effective amount of an anticoagulant, and wherein the patient does not receive any co-treatment with an anticoagulant.

2. The composition for use according to claim 1, wherein said cells express at least one hepatic marker and/or exhibit a liver-specific activity.

3. The composition for use according to claim 1, wherein said cells secrete HGF.

4. The composition for use according to claim 1, wherein said cells secrete HGF and PGE2.

5. The composition for use according to claim 1, wherein said cells are measured:
a. positive for α-smooth muscle actin (ASMA), CD140b and optionally albumin (ALB);
b. negative for Cytokeratin-19 (CK-19) and optionally for Sushi domain containing protein 2 (SUSD2).

6. The composition for use according to claim 1, wherein said cells are further measured positive for:
a. At least one hepatic marker selected from HNF-3B, HNF-4, CYP1A2, CYP2C9, CYP2E1 and CYP3A4 and optionally albumin;
b. At least one mesenchymal marker selected from Vimentin, CD90, CD73, CD44, and CD29;
c. At least one liver-specific activity selected from urea secretion, bilirubin conjugation, alpha-1-antitrypsin secretion, and CYP3A4 activity;
d. At least one marker selected from ATP2B4, ITGA3, TFRC, SLC3A2, CD59, ITGB5, CD151, ICAM1, ANPEP, CD46, and CD81; and
e. At least one marker selected from MMP1, ITGA11, FMOD, KCND2, CCL11, ASPN, KCNK2, and HMCN1.

7. The composition for use according to claim 1, wherein the composition comprises a dose of 0.5 to 1 million HALPC cells per kg body weight.

8. The composition for use according to claim 1, wherein the patient has been or is diagnosed with a disease or condition selected from a non-cirrhotic chronic liver disease, cirrhosis, compensated cirrhosis, decompensated cirrhosis (DC), acute decompensated cirrhosis, acute decompensation (AD), and optionally wherein the patient is pre-ACLF or ACLF grade-0, or has an ACLF grade level selected from ACLF-1 and ACLF-2.

9. The composition for use according to claim 1, wherein the patient, prior to the treatment, has been diagnosed with a MELD score in the range from 13 to 35, and/or is experiencing or has experienced at least one organ failure.

10. The composition for use according to claim 1, wherein the patient, prior to the treatment, exhibits a total bilirubin serum concentration of at least 5 mg/dL, or of at least 6 mg/dL.

11. The composition for use according to claim 1, wherein the composition is administered to the patient in the form of a sterile liquid comprising the HALPC cells at a concentration of 0.5 to 5 million cells per mL.

12. The composition for use according to claim 11, wherein the sterile liquid composition is intravenously infused to the patient at an infusion rate of about 0.1 to about 5 mL per minute, or at a rate of 0.5 to about 2 mL per minute, such as about 1.5 mL per minute.

13. The composition for use according to claim 12, wherein the composition is administered to the patient a vertically mounted infusion pump.

14. The composition for use according to claim 1, wherein the treatment further comprises:
administering to said patient a second amount of said composition, said second amount comprising a second dose of 0.25 to 2.5 million HALPC cells per kg body weight;
wherein said second amount is administered 5 to 21 days after the first amount;
wherein said composition is substantially free of an effective amount of an anticoagulant, and wherein the patient does not receive any co-treatment with an anticoagulant.

15. The composition for use according to claim 14, wherein said second amount is administered 6 to 8 days after said first amount.

16. The composition for use according to claim 15, wherein said second amount comprises a second dose of 0.5 to 1 million HLAPC cells per kg body weight.

17. The composition for use according to claim 15 or 16, wherein said second amount is administered 7 days after said first amount.

18. The composition for use according to claim 1, wherein the treatment results in a decrease in MELD score of the patient by at least 20%, preferably within 28 days after the composition is first administered to the patient.
